(19) United States
(12) Patent Application Publication
(10) Pub. No.: US 2009/0118258 A1
(43) Pub. Date: May 7, 2009

(54) COMBINATION THERAPY COMPRISING ANGIOTENSIN CONVERTING ENZYME INHIBITORS AND VASOPRESSIN RECEPTOR ANTAGONISTS

(76) Inventors: Bruce Damiano, Warrington, PA (US); Lloyd Haskell, Flemington, NJ (US); Umesh Shukla, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/265,098

(22) Filed: Nov. 5, 2008

Related U.S. Application Data
(60) Provisional application No. 60/986,103, filed on Nov. 7, 2007.

(51) Int. Cl. A61K 31/55; A61P 9/04; A61P 9/12; A61P 9/10; A61P 25/00

(52) U.S. Cl. 514/212.02

(57) ABSTRACT

The present invention relates to certain pharmaceutical compositions containing at least one vasopressin receptor antagonist and at least one angiotensin converting enzyme (ACE) inhibitor and methods for preparing these compounds, compositions, intermediates and derivatives thereof and for the treatment of vasopressin and/or angiotensin converting enzyme mediated disorders.

### CROSS REFERENCE TO RELATED APPLICATION

This application claims the benefit of U.S. Provisional Application No. 60/986,103 filed Nov. 7, 2007, which is incorporated by reference herein.

### FIELD OF THE INVENTION

The present invention relates to pharmaceutical compositions containing at least one vasopressin receptor antagonist and at least one angiotensin converting enzyme (ACE) inhibitor. The invention also relates to methods of treating, ameliorating and/or preventing the progression of vasopressin and/or angiotensin converting enzyme mediated diseases including, but not limited to, diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors comprising administering such pharmaceutical compositions to human patients.

### BACKGROUND OF THE INVENTION

Vasopressin is a nonapeptide hormone that is secreted primarily from the posterior pituitary gland. The hormone effects its actions through the V1a and V2 receptor subtypes. The functions of vasopressin include contraction of uterine, bladder, and vascular smooth muscle; stimulation of glycogen breakdown in the liver; induction of platelet aggregation; release of corticotropin from the anterior pituitary and stimulation of renal water reabsorption. As a neurotransmitter within the central nervous system (CNS), vasopressin can affect aggressive behavior, sexual behavior, the stress response, social behavior and memory. The V1a receptor mediates central nervous system effects, contraction of smooth muscle and hepatic glycogenolytic effects of vasopressin, while the V1b receptor mediates anterior pituitary effects of vasopressin. The V2 receptor, presumably found only in the kidney, effects the antidiuretic actions of vasopressin via stimulation of intracellular adenylate cyclase (Liebsch, G et al Neurosci. 1996, 217, 101).

Elevated plasma vasopressin levels appear to play a role in the pathogenesis of congestive heart failure (P. A. Van Zwieten, Progr. Pharmacol. Clin. Pharmacol. 1990, 7, 49). As progress toward the treatment of congestive heart failure, nonpeptide vasopressin V2 receptor antagonists have induced low osmolality aquaresis and decreased peripheral resistance in conscious dogs with congestive heart failure (H. Ogawa, J. Med. Chem. 1996, 39, 3547). In certain pathological states, plasma vasopressin levels may be inappropriately elevated for a given osmolality, thereby resulting in renal water retention and hyponatremia. Hyponatremia, associated with edematous conditions (cirrhosis, congestive heart failure, renal failure), can be accompanied by the syndrome of inappropriate secretion of antidiuretic hormone (SIADH). Treatment of SIADH-compromised rats with a vasopressin V2 antagonist has corrected their existing hyponatremia (G. Fujisawa, Kidney Int. 1993, 44(1), 19). Due in part to the contractile actions of vasopressin at its V1a receptor in the vasculature, vasopressin V1a antagonists have reduced blood pressure and represent a potential treatment for hypertension as well. Known vasopressin receptor antagonists have included YM-087 (Yamanouchi); VPA-985, WAY-140288, and CL-385004 (American Home Products); SR-121463 (Sanofi-Synthelabo); and OPC 31260, OPC 41061, and OPC 21268 (Otsuka).

Thus, vasopressin receptor antagonists are useful as therapeutics in the conditions of hypertension, hyponatremia, congestive heart failure/cardiac insufficiency, coronary vasospasm, cardiac ischemia, liver cirrhosis, renal vasospasm, renal failure, diabetic nephropathy, cerebral edema and ischemia, stroke, thrombosis, and water retention. Additional conditions may include nephrotic syndrome, central nervous system injuries, dysmenorrhea, aggression, polycystic kidney diseases, anxiety, obsessive-compulsive disorders and other diseases resulting from excessive activation of vasopressin V1a and V2 receptors.

Particularly, nephropathyand renal failure are common complications of long-standing diabetes and/or hypertension. It has been well documented that maintenance of tight glycemic control and adequate control of hypertension, in combination with the administration of an angiotensin converting enzyme (ACE) inhibitor or angiotensin receptor antagonist, can slow disease progression. Despite this standard care, a significant risk and incidence of progression to renal failure remains. Thus, there is a significant unmet medical need for novel treatments to further slow the progression of this disease.

The most well-accepted theory of progressive nephropathy in humans involves an initial reduction in nephron number due to varying pathological insults (eg. diabetes, inflammation, etc), which leads to damage of remaining functioning nephrons as a consequence of adaptive increases in glomerular pressure and flow to compensate for loss of nephron function (Anderson S, Meyer T W, Brenner B M: The role of hemodynamic factors in the initiation and progression of renal disease. J Urol 133:363 368,1985; Remuzzi G, Bertani T: Pathophysiology of progressive nephropathies. N Engl J Med 339: 1448 1456,1998.). The glomerular hypertension responsible for maintaining the necessary hyperfiltration in these initially healthy nephrons is accompanied by enhanced filtration of plasma proteins that, being largely reabsorbed by a process of tubular endocytosis, exert a nephritogenic effect that would favor tissue scarring and functional impairment. Thus, the rat remnant kidney model, which involves the artificial loss of nephrons induced by removal of one kidney and damage of a portion of the remaining kidney, represents a good model to simulate the processes and pathology that occur in human nephropathy (Olson J L, Hostetter T H, Rennke H G, Brenner B M, Venkatachalam M A: Altered glomerular permselectivity and progressive sclerosis following extreme ablation of renal mass. Kidney Int 22:112 126,1982). Moreover the model has good clinical validation with respect to treatments for renal failure. Importantly, inhibition of angiotensin converting enzyme (ACE) (Anderson S, Rennke H G, Brenner B M: Therapeutic advantage of converting enzyme inhibitors in arresting progressive renal disease associated with systemic hypertension in the rat. J Clin Invest 77:1993 2000,1986. ) and angiotensin receptor blockade (Tarif N and Bakris G L: Angiotensin II receptor blockade and progression of nondiabetic-mediated renal disease. Kidney Int. 52: S67-S70,1997) have been shown to be effective in the remnant kidney model (as evidenced by reduction in urine protein, serum creatinine and glomerular sclerosis) and, were subsequently shown to reduce urine protein in patients with diabetic nephropathy as well as improve other measures of renal function such as serum creatinine (Kshirsagar, A V, Joy, M S, Hogan, S L, Falk, R J & Colindres, R E: Effect of ACE inhibitors in diabetic and nondiabetic chronic renal disease: a systematic overview of randomized placebo-controlled trials. Am. J. Kidney Dis, 35: 695-707, 2000; Coyle, J D, Gardner, S F & White, C M: The renal protective effects of angiotensin II receptor blockers in type 2 diabetes mellitus. Annals Pharmacotherapy, 38:1731-8, 2004). In addition, clinical studies have shown that treatment with ACE inhibitors and angiotensin receptor blockers increases time to renal failure (need for dialysis or kidney transplant). Thus, reduction in urine albumen is a good surrogate for predicting impact on disease progression independent of treatment mechanisms.

Accordingly, there is a need for a therapeutically effective pharmaceutical composition comprising at least one vasopressin receptor antagonist and at least one angiotensin converting enzyme inhibitor. There is also a need for an effective method for treating, ameliorating, and/or slowing the progression of vasopressin and/or ACE mediated disorders.

### SUMMARY OF THE INVENTION

The present invention is directed to a pharmaceutical composition comprising at least one angiotensin-converting enzyme inhibitor, at least one vasopressin receptor antagonist, and a pharmaceutically acceptable carrier.

The present invention is also directed to a method for treating, ameliorating, and/or slowing the progression of vasopressin and/or ACE mediated disorders including but not limited to diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors, or associated symptoms or complications thereof in a subject, said method comprising administering to said subject a therapeutically effective amount of at least one angiotensin-converting enzyme inhibitor and administering to said subject a therapeutically effective amount, of at least one vasopressin receptor antagonist, said combined administration providing the desired therapeutic effect.

In the disclosed embodiments of the invention, the vasopressin and/or ACE mediated disorders, or associated symptoms or complications thereof, are selected from diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors. Preferably, the therapeutically effective amount of the compound administered for treating any of these conditions is about 0.05 to 1 g per day.

The present invention is still further directed to the use of one or more angiotensin-converting enzyme inhibitors in combination with one or more vasopressin receptor antagonists for the preparation of a medicament for treating, ameliorating, and/or slowing the progression of a condition selected from diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors.

Other features and advantages of the invention will become apparent from the detailed disclosure, the examples, and the appended claims.

### BRIEF DESCRIPTION OF THE FIGURES

FIG. 1 shows the marked increase in urine protein and serum creatinine 21 days following RMR in the vehicle-treated animals, as well as the progressive increase in these measures at 1 and 2 months.

FIG. 2 shows the measure of structural damage based on renal histology in the rat remnant kidney model at the end of the experiment.

FIG. 3 shows the blood pressure values in the rat remnant kidney model.

FIG. 4 shows the urine albumin excretion in Streptozotocin-induced Type I diabetic rat.

FIG. 5 shows urine albumin excretion in the db/db mouse model of type II diabetes.

### DETAILED DESCRIPTION OF THE INVENTION

Unless indicated otherwise, the following definitions apply throughout the present specification and claims.

“At least one” means one or more (e.g., 1-3, 1-2, or 1).

“Composition” is intended to encompass a product comprising the specified ingredients in the specified amounts, as well as any product which results, directly or indirectly, from combination of the specified ingredients in the specified amounts.

“In combination with” as used to describe the administration of a compound of formula I with other medicaments in the methods of treatment of this invention, means that the compounds of formula I and the other medicaments are administered sequentially or concurrently in separate dosage forms, or are administered concurrently in the same dosage form.

“Mammal” includes a human being, and preferably means a human being.

“Patient” includes both human and other mammals, preferably human.

“Alkyl” means a straight or branched saturated hydrocarbon chain having 1 to 20 carbon atoms, preferably 1 to 12 carbon atoms, more preferably 1 to 6 carbon atoms.

“Alkoxy” means an alkyl-O-group wherein alkyl is as defined above. Non-limiting examples of alkoxy groups include: methoxy, ethoxy, n-propoxy, iso-propoxy and n-butoxy. The bond to the parent moiety is through the ether oxygen.

In one embodiment of the present invention, a pharmaceutical composition comprises at least one angiotensin-converting enzyme inhibitor, at least one vasopressin V1a/V2 receptor antagonist, and a pharmaceutically acceptable carrier.

Many ACE inhibitors can be employed in this invention. The ACE inhibitors to be used in the compositions of this invention are well known in the art, and several are used routinely for treating hypertension, diabetic nephropathy and chronic heart failure. For example, enalapril, enalaprilat, and closely related analogs are described in U.S. Pat. Nos. 4,374,829; 4,472,380; and 4,264,611. Moexipril, quinapril, quinaprilat, and related analogs are described in U.S. Pat. Nos. 4,743,450 and 4,344,949. Ramipril and its analogs are described in U.S. Pat. Nos. 4,587,258 and 5,061,722. Lisinopril is described in U.S. Pat. No. 4,374,829. All of the foregoing patents are incorporated herein by reference for their teaching of typical ACE inhibitors.

In one embodiment of the present invention, the ACE inhibitor is selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril. In a further embodiment of the present invention, the ACE inhibitor is enalapril. In another embodiment of the present invention, the ACE inhibitor is lisinopril.

The vasopressin antagonist of the present invention is defined as any chemical compound that is effective in inhibiting the biological activity of any arginine vasopressin or antidiuretic hormone.

In one embodiment of the present invention, the vasopressin antagonist is a compound of Formula (I)

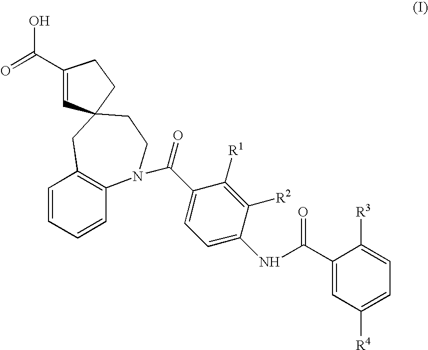

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof. (See U.S. patent application Ser. No. 10/869,746)

An embodiment of the present invention is further directed to a vasopressin antagonist of Compound 1

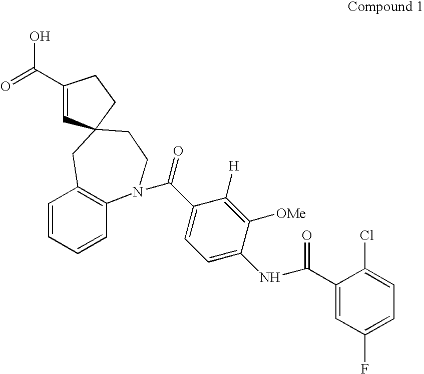

or a pharmaceutically acceptable C1-6 ester, C1-6 amide, or di(C1-6 alkyl)amide or salt thereof.

In another embodiment of the present invention, the pharmaceutical composition comprises at least one angiotensin-converting enzyme inhibitor selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril and at least one vasopressin antagonist selected from Formula (I)

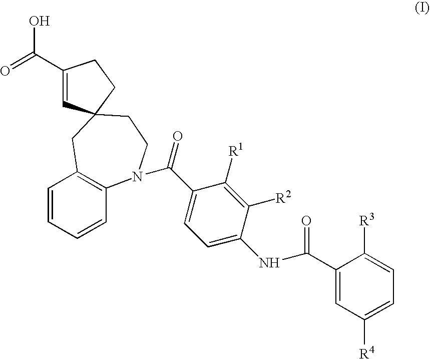

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, and a pharmaceutically acceptable carrier.

In a further embodiment of the present invention, the pharmaceutical composition comprises at least one angiotensin-converting enzyme inhibitor selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril and at least one vasopressin antagonist which is

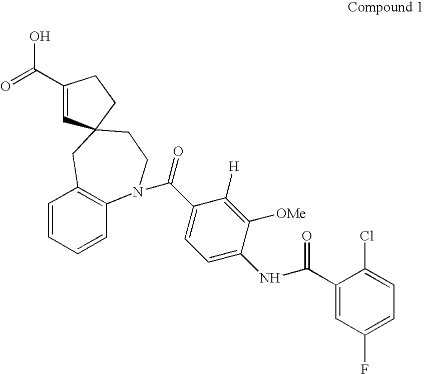

or a pharmaceutically acceptable C1-6 ester, C1-6 amide, or di(C1-6 alkyl)amide or salt thereof, and a pharmaceutically acceptable carrier.

In still a further embodiment of the present invention, the pharmaceutical composition comprises at least one angiotensin-converting enzyme inhibitor selected from the group consisting of enalapril and lisinopril and at least one vasopressin antagonist selected from Formula (I)

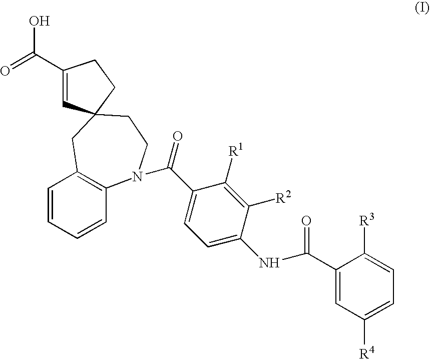

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, and a pharmaceutically acceptable carrier.

In still a further embodiment of the present invention, the pharmaceutical composition comprises at least one angiotensin-converting enzyme inhibitor wherein such angiotensin-converting enzyme inhibitor is lisinopril and at least one vasopressin antagonist selected from Formula (I)

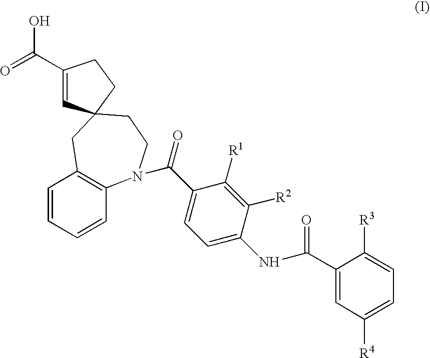

wherein

one of R¹ and R² is H and the other is C₁₋₆ alkoxy;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, and a pharmaceutically acceptable carrier.

In a still further embodiment of the present invention, the pharmaceutical composition comprises at least one angiotensin-converting enzyme inhibitor selected from the group consisting of enalapril and lisinopril, and at least one vasopressin antagonist which is

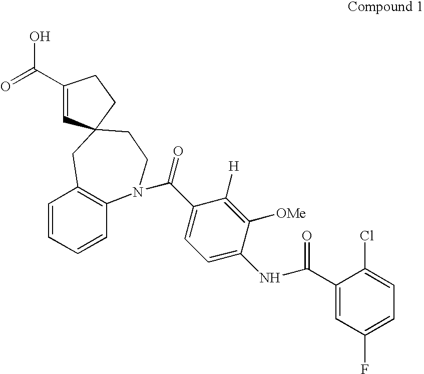

or a pharmaceutically acceptable C1-6 ester, C1-6 amide, or di(C1-6 alkyl)amide or salt thereof, and a pharmaceutically acceptable carrier.

In a still further embodiment of the present invention, the pharmaceutical composition comprises at least one angiotensin-converting enzyme inhibitor which is enalapril, and at least one vasopressin antagonist which is

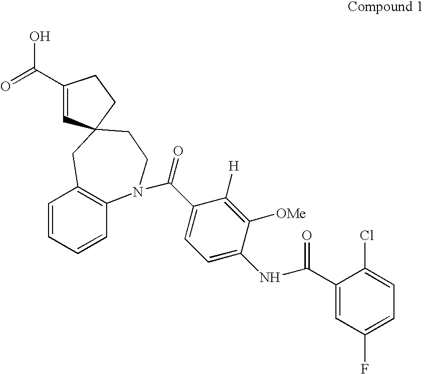

or a pharmaceutically acceptable C1-6 ester, C1-6 amide, or di(C1-6 alkyl)amide or salt thereof, and a pharmaceutically acceptable carrier.

In yet still another such embodiment of the present invention, the pharmaceutical composition comprises at least one angiotensin-converting enzyme inhibitor, which is lisinopril, and at least one vasopressin antagonist is

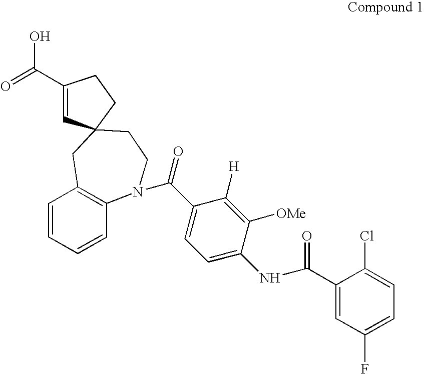

or a pharmaceutically acceptable C1-6 ester, C1-6 amide, or di(C1-6 alkyl)amide or salt thereof, and a pharmaceutically acceptable carrier.

In one embodiment of the invention, there is disclosed a method for treating a vasopressin and/or ACE mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

In one embodiment of the invention, there is disclosed a method for treating a vasopressin mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

In one embodiment of the invention, there is disclosed a method for treating an ACE mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

In a further embodiment of the present invention, the method comprises administering to said subject a therapeutically effective amount of at least one angiotensin-converting enzyme inhibitor selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril, in combination with at least one vasopressin antagonist selected from Formula (I)

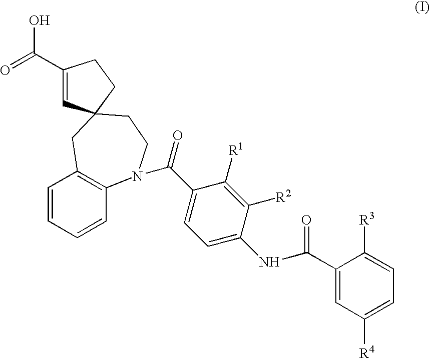

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

In still a further embodiment of the present invention, the method comprises administering to said subject a therapeutically effective amount of at least one angiotensin-converting enzyme inhibitor selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril, in combination with at least one vasopressin antagonist wherein such antagonist is

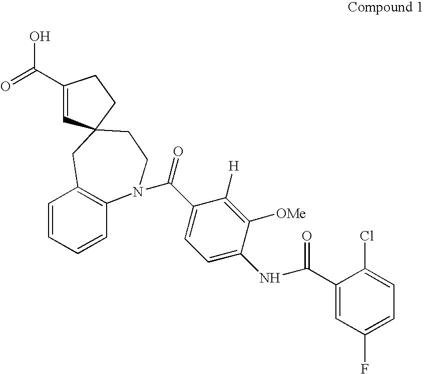

or a pharmaceutically acceptable C1-6 ester, C1-6 amide, or di(C1-6 alkyl)amide or salt thereof, and a pharmaceutically acceptable carrier said combined administration providing the desired therapeutic effect.

In yet a further embodiment of the present invention, the method comprises administering to said subject a therapeutically effective amount of at least one angiotensin-converting enzyme inhibitor selected from the group consisting of enalapril and lisinopril in combination with at least one vasopressin receptor antagonist selected from Formula (I)

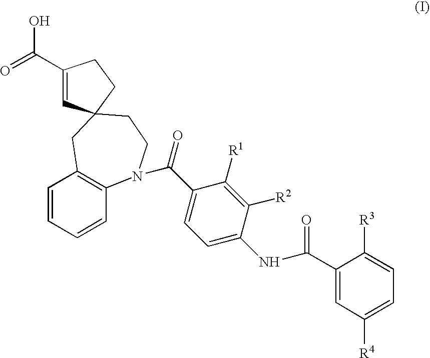

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

In a further embodiment of the present invention, the method comprises administering to said subject a therapeutically effective amount of at least one angiotensin-converting enzyme inhibitor selected from the group consisting of enalapril and lisinopril, in combination with at least one vasopressin antagonist wherein such antagonist is

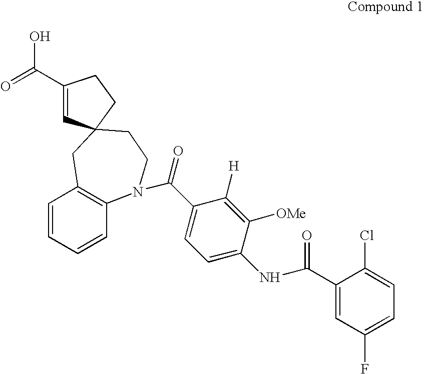

or a pharmaceutically acceptable C1-6 ester, C1-6 amide, or di(C1-6 alkyl)amide or salt thereof, and a pharmaceutically acceptable carrier said combined administration providing the desired therapeutic effect.

In a further embodiment of the present invention, the method comprises administering to said subject a therapeutically effective amount of at least one angiotensin-converting enzyme inhibitor wherein such inhibitor is lisinopril, in combination with at least one vasopressin antagonist wherein such antagonist is

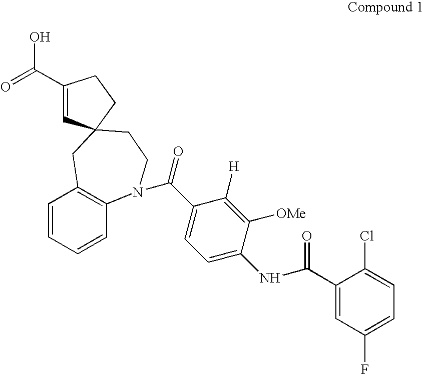

or a pharmaceutically acceptable C1-6 ester, C1-6 amide, or di(C1-6 alkyl)amide or salt thereof, and a pharmaceutically acceptable carrier said combined administration providing the desired therapeutic effect.

In another embodiment of the present invention, there is a method for inhibiting or slowing the progression of a vasopressin an/or ACE mediated disorder or associated symptoms or complications thereof in a subject, said method comprising administering to said subject a therapeutically effective amount of at least one angiotensin-converting enzyme inhibitor in combination with at least one vasopressin antagonist, said combined administration providing the desired prophylactic effect.

In one such embodiment of the present invention, said method comprising administering to said subject a therapeutically effective amount of at least one angiotensin-converting enzyme inhibitor is selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril in combination with at least one vasopressin antagonist selected from Formula (I)

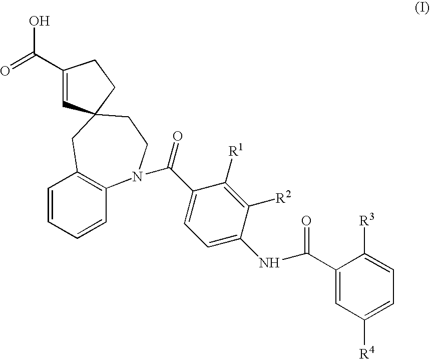

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R⁴ is chloro, fluoro, methoxy, or methyl;

or a pharmaceutically acceptable C₁₋₆ ester, C₁₋₆ amide, or di(C₁₋₆ alkyl)amide or salt thereof, said combined administration providing the desired prophylactic effect.

In another such embodiment of the present invention, said method comprising administering to said subject a therapeutically effective amount of at least one angiotensin-converting enzyme inhibitor is selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril in combination with at least one vasopressin antagonist is a compound of

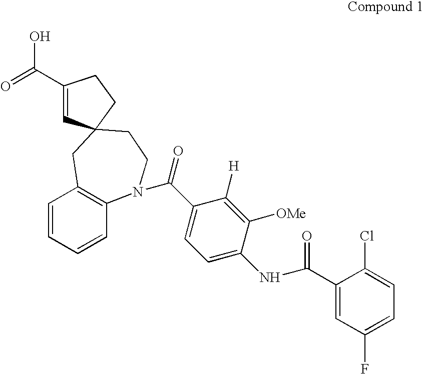

or a pharmaceutically acceptable C1-6 ester, C1-6 amide, or di(C1-6 alkyl)amide or salt thereof said combined administration providing the desired prophylactic effect.

In still another such embodiment of the present invention, said method comprising administering to said subject a therapeutically effective amount of at least one angiotensin-converting enzyme inhibitor is selected from the group consisting of enalapril and lisinopril in combination with at least one vasopressin antagonist selected from Formula (I)

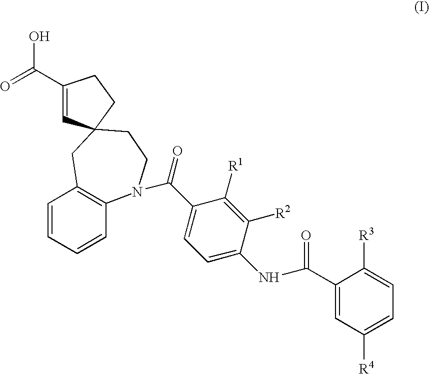

wherein

one of R¹ and R² is H and the other is H, NR⁵R⁶, C₁₋₆ alkoxy, hydroxy, or halo; wherein each of R⁵ and R⁶ is independently H or C₁₋₃ alkyl;

R³ is chloro;

R4 is chloro, fluoro, methoxy, or methyl;

or a pharmaceutically acceptable C₁₋₆ ester, C₁₋₆ amide, or di(C₁₋₆ alkyl)amide or salt thereof, said combined administration providing the desired prophylactic effect.

In yet another such embodiment of the present invention, said method comprising administering to said subject a therapeutically effective amount of at least one angiotensin-converting enzyme inhibitor is selected from the group consisting of enalapril and lisinopril in combination with at least one vasopressin receptor antagonist is a compound of Formula (I)

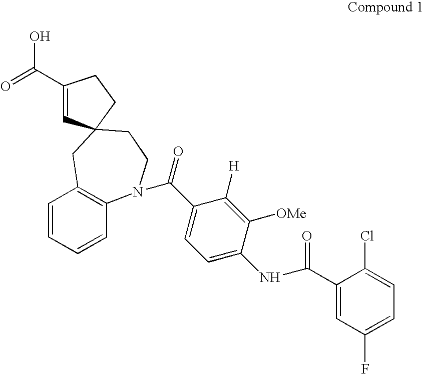

or a pharmaceutically acceptable C1-6 ester, C1-6 amide, or di(C1-6 alkyl)amide or salt thereof, said combined administration providing the desired prophylactic effect.

In yet still another such embodiment of the present invention, said method comprising administering to said subject a therapeutically effective amount of at least one angiotensin-converting enzyme inhibitor wherein said inhibitor is lisinopril in combination with at least one vasopressin receptor antagonist wherein such antagonist is

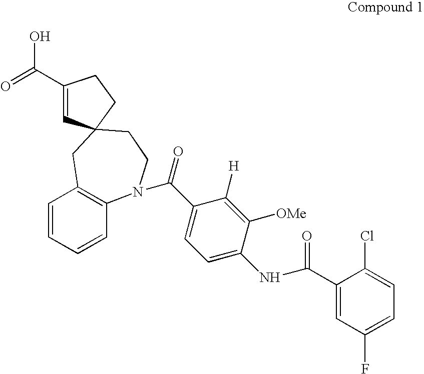

or a pharmaceutically acceptable C1-6 ester, C1-6 amide, or di(C1-6 alkyl)amide or salt thereof, said combined administration providing the desired prophylactic effect.

In one embodiment of the invention, said disorder is selected from disease states of inner ear disorders, hypertension, congestive heart failure, cardiac insufficiency, hyponatremia, coronary vasospasm, cardiac ischemia, liver cirrhosis, renal vasospasm, renal failure, diabetic nephropathy, polycystic kidney disease, cerebral edema and ischemia, stroke, thrombosis, water retention, aggression, obsessive-compulsive disorders, dysmenorrhea, nephrotic syndrome, and central nervous injuries.

In another embodiment of the invention, said disorder is selected from diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors.

In one embodiment the disorder is nephropathy. In another embodiment the disorder is progressive renal failure. In yet another embodiment the disorder is diabetic nephropathy. In still another embodiment, the disorder is polycystic kidney disease. In yet still another embodiment, the disorder is congestive heart failure. In a further embodiment the disorder is hypertension. In yet a further embodiment the disorder is hyponatremia. In still a further embodiment the disorder is edema. In yet still a further embodiment the disorder results from excessive activation of vasopressin V1a and V2 receptors.

In an embodiment of the present invention, there is disclosed a process for formulating a pharmaceutical composition, comprising formulating together at least one angiotensin-converting enzyme inhibitor, at least one vasopressin antagonist, and a pharmaceutically acceptable carrier.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Even though the compounds of the present invention (including their pharmaceutically, acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention is directed to pharmaceutical and veterinary compositions comprising the combination of a compound of Formula (I) and an ACE inhibitor, along with one or more pharmaceutically acceptable carriers, excipients or diluents.

By way of example, in the pharmaceutical and veterinary compositions of the present invention, the compounds of the present invention may be admixed with any suitable binder(s), lubricant(s), suspending agent(s), coating agent(s), and/or solubilising agent(s).

Tablets or capsules of the combination may be administered singly or two or more at a time, as appropriate. It is also possible to administer the compounds in sustained release formulations.

Alternatively, the combination of a compound of the general Formula (I) and an ACE inhibitor can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

For some applications, preferably the combined compositions are administered orally in the form of tablets containing excipients such as starch or lactose, or in capsules or ovules either alone or in admixture with excipients, or in the form of elixirs, solutions or suspensions containing flavoring or coloring agents.

The combined compositions (as well as the compounds alone) can also be injected parenterally, for example intracavernosally, intravenously, intramuscularly or subcutaneously. In this case, the combined compositions will comprise a suitable carrier or diluent.

For parenteral administration, the combined compositions are best used in the form of a sterile aqueous solution which may contain other substances, for example enough salts or monosaccharides to make the solution isotonic with blood.

For buccal or sublingual administration the combined compositions may be administered in the form of tablets or lozenges which can be formulated in a conventional manner.

By way of further example, pharmaceutical and veterinary compositions containing the combined compounds of the invention described herein as the active ingredient can be prepared by intimately mixing compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, the combined compounds of the present invention may be administered in a single daily dose, or the total daily dosage may be administered in divided doses of two, three or four times daily. Furthermore, the combined compounds for the present invention can be administered in intranasal form via topical use of suitable intranasal vehicles, or via transdermal skin patches well known to those skilled in that art. To be administered in the form of a transdermal delivery system, the dosage administration will, of course, be continuous rather than intermittent throughout the dosage regimen.

As used herein, a “therapeutically effective amount” is that quantity that gives a positive effect in treating, ameliorating or slowing the progession of any one of the vasopressin and/or ACE mediated disorders. For example, a therapeutically effective amount is that quantity that gives a positive effect in treating diabetic nephropathy and progressive renal failure by causing a reduction in urinary protein. The composition of this invention will contain an ACE inhibitor and a vasopressin antagonist in a weight ratio of about 1 to about 200 particularly about 5 to about 100, and even more particularly about 10 to about 50. Typical effective amounts will be about 4 to about 50 mg of ACE inhibitor, and about 10 to about 800 mg of vasopressin antagonist.

The precise dosage that is effective according to this invention is to be determined by the attending medical practitioner, taking into account the specific ACE inhibitor and vasopressin antagonist being administered, the particular condition of the subject being treated, the duration of the treatment and severity of the disease, and such other factors routinely considered when practicing sound medical judgment. The therapeutically effective amount for administering the pharmaceutical composition to a human, for example, can be determined mathematically from the results of animal studies.

A therapeutically effective amount for use of the instant invention or a pharmaceutical composition thereof comprises a dose range from about 0.1 mg to about 3000 mg, in particular from about 1 mg to about 1000 mg or, more particularly from about 10 mg to about 500 mg of active ingredient in a regimen of about 1 to 4 times per day for an average (70 kg) human; although, it is apparent to one skilled in the art that the therapeutically effective amount for active compounds of the invention will vary as will the conditions being treated.

For oral administration, a pharmaceutical composition is preferably provided in the form of tablets containing 0.01, 0.05, 0.1, 0.5, 1.0, 2.5, 5.0, 10.0, 15.0, 25.0, 50.0, 100, 150, 200, 250, and 500 milligrams of the active ingredient for the symptomatic adjustment of the dosage to the subject to be treated.

It is also apparent to one skilled in the art that the therapeutically effective dosages of the combination of an ACE inhibitor with a compound of Formula (I) to be administered for the treatment of or prevention of vasopressin and/or ACE-mediated disorders may be readily determined by those skilled in the art, and will vary according to the desired effect. Therefore, optimal dosages to be administered may be readily determined and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

Compounds of this invention may be administered in any of the foregoing compositions and dosage regimens or by means of those compositions and dosage regimens established in the art whenever use of the compounds of the invention is required for a subject in need thereof.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### BIOLOGICAL EXPERIMENTAL EXAMPLES

As demonstrated by biological studies described hereinafter, and shown in Table I and Table II, the compounds of the present invention—vasopressin antagonists and ACE inhibitors—may be useful in treating inner ear disorders, hypertension, congestive heart failure, cardiac insufficiency, hyponatremia, coronary vasospasm, cardiac ischemia, liver cirrhosis, renal vasospasm, renal failure, diabetic nephropathy, polycystic kidney disease, cerebral edema and ischemia, stroke, thrombosis, water retention, aggression, obsessive-compulsive disorders, dysmenorrhea, nephrotic syndrome, and central nervous injuries.

More particularly compounds of the present invention—vasopressin antagonists and ACE inhibitors—may be useful in treating diabetic nephropathy, progressive renal failure, polycystic kidney diseases, congestive heart failure, hypertension, diseases resulting in hyponatremia and/or edema, and other diseases resulting from excessive activation of vasopressin V1a and V2 receptors.

### Example 1

### Efficacy in Rat Remnant Kidney Model of Nephropathy

The rat remnant kidney model is an animal model of severe progressive renal failure. In this model, a state of severe progressive renal failure is produced by renal mass reduction (RMR) through removal of one kidney and ligation of several branches of the renal artery, resulting in infarction and loss of function of two thirds of the remaining kidney. The procedure creates the conditions of severe hypertension, proteinuria, progressive renal function deterioration, tubulointerstitial damage and glomerulosclerosis (Olson J L, Hostetter T H, Rennke H G, Brenner B M, Venkatachalam M A: “Altered glomerular permselectivity and progressive sclerosis following extreme ablation of renal mass”. Kidney Int 22:112-126, 1982).

Nephropathy is evident as a progressively increasing amount of protein in urine, increases in serum creatinine and increases in arterial pressure. In addition, severe sclerosis of the glomeruli, inflammatory cell infiltration and tubular damage are measurable through histological analysis within 3 months of renal mass reduction (RMR). These measures form the basis for determining efficacy in the model. Urine protein, serum creatinine and arterial pressure are similarly measured in humans to determine impact of treatments on disease progression.

The aim of this study was to evaluate the effects of Compound 1 compared to an ACE inhibitor (enalapril), as well as the combination treatment with Compound 1 and enalapril, on proteinuria and renal disease progression in this model. The effectiveness of these agents has been associated with reductions in proteinuria, slowing of the rate of rise in serum creatinine and reduction in glomerular sclerosis and thus slowing of the progression to end stage renal failure.

Treatments were started 21 days after RMR, when animals already had overt nephropathy. Treatment groups (n=8/group, except enalapril n=7) included vehicle; Compound 1 at 10 mg/kg/d; Compound 1 at 30 mg/kg/d; enalapril at 15 mg/mL (in drinking water); and enalapril at 15 mg/mL+Compound 1 at 30 mg/kg/d. All compounds were administered in drinking water. An additional sham-operated untreated group was also included. Measurements were made at 21 d after RMR but before treatment, and at one and two months of treatment. An apparent excess mortality occurred in the 10 and 30 mg/kg/d Compound 1 treatment groups. Since the increased mortality was not dose dependent and was not apparent in the group receiving combination treatment with Compound 1 and enalapril (Table 1), the effect is likely due to chance and the known variability in the severity of renal failure in this model.

TABLE 1
Mortality Rates in Remnant Kidney Model

| | 1 month | 2 month |
|---|---|---|
| Vehicle | 1/8 | 2/8 |
| Compound 1, 10 mg/kg/d | 2/8 | 4/8 |
| Compound 1, 30 mg/kg/d | 2/8 | 3/8 |
| Enalapril, 15 mg/mL | 0/7 | 1/7 |
| Compound 1, 30 mg/kg/d + enalapril 15 mg/mL | 2/8 | 2/8 |

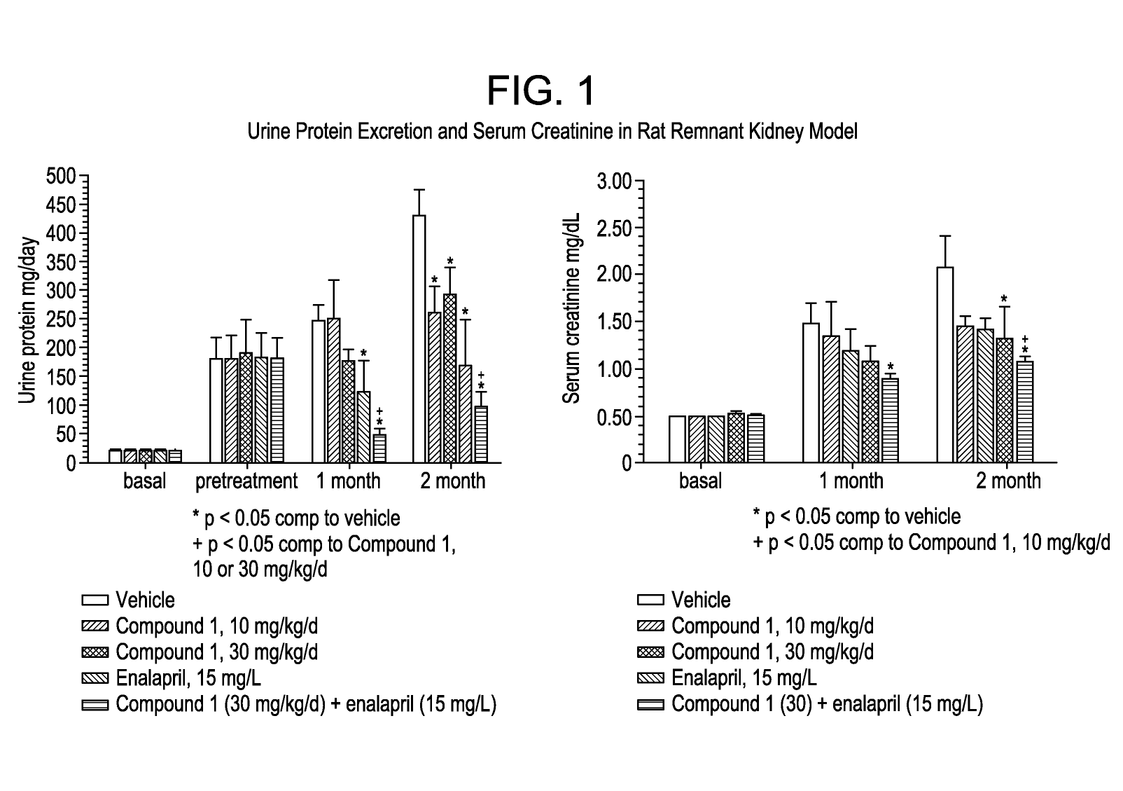

FIG. 1 shows the marked increase in urine protein and serum creatinine 21 days following RMR in the vehicle-treated animals, as well as the progressive increase in these measures at 1 and 2 months. Enalapril significantly reduced urine protein and serum creatinine at both one and two months of treatment. Compound 1 (10 and 30 mg/kg/d), also reduced urine protein and serum creatinine when administered alone but not to the same extent as enalapril. However, combination treatment with enalapril and Compound 1 (30 mg/kg/d) reduced urine protein and serum creatinine to a greater extent than enalapril alone. This finding is significant in that the two mechanisms for nephroprotection i.e., inhibition of angiotensin converting enzyme and inhibition of vasopressin receptors are independent and that the use of both compounds can be combined to produce incremental benefit in human nephropathy.

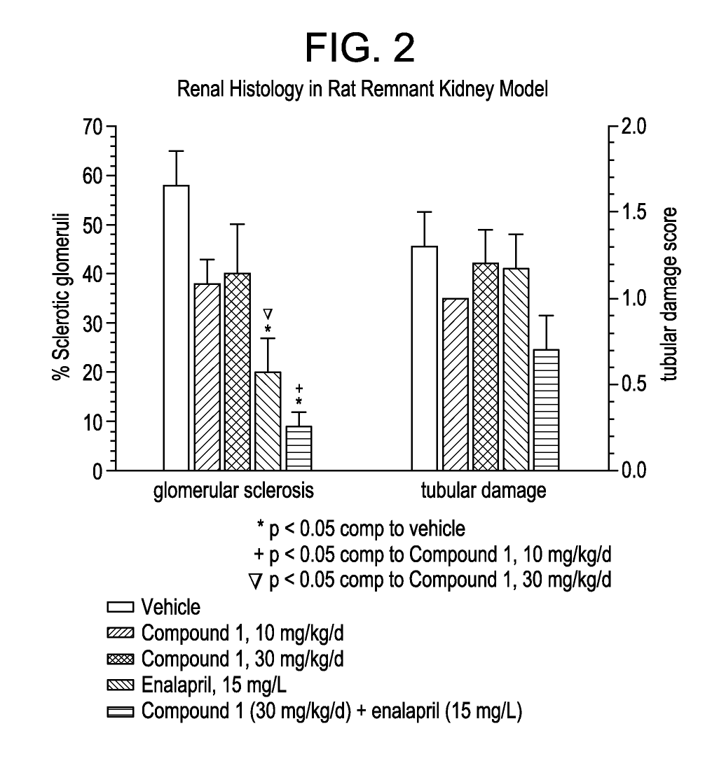

At the termination of the experiment kidneys were examined quantitatively for histopathological damage. Nearly 60% of glomeruli from vehicle-treated rats were sclerotic (FIG. 2). Enalapril treatment produced a significant reduction in the percentage of sclerotic glomeruli compared to vehicle-treated rats. Compound 1 at 10 or 30 mg/kg/d tended to decrease the percentage of sclerotic glomeruli compared with vehicle, but this was not statistically significant. Importantly, combination treatment with enalapril and Compound 1, 30 mg/kg/d, produced a further decrease in sclerosis compared to enalapril alone. There were no statistically significant effects on tubular damage score in any treatment group. However, there was a trend for reduction in tubular damage score in the combination treatment group. It is notable that the degree of tubular damage in the combination treatment group, in contrast to the other treatment groups, was not statistically different from the tubular damage values in normal kidneys. Accumulation of monocytes/macrophages in the renal interstitium was quantified as the number of ED 1 positive cells per field. The number of positive cells was significantly greater in the vehicle treated rats with RMR (63±4) compared with control rats. Compound 1 tended to reduce accumulation of monocytes/macrophages in the renal interstitium at 10 and 30 mg/kg/d (48±4 and 49±10, respectively), although this was not statistically significant. However, enalapril (38±6) and the combination of enalapril and Compound 1 30 mg/kg/d (29±5) caused statistically significant reduction in cell number compared with vehicle. Cell reduction tended to be greater in the combination treatment group.

The histological data indicate that the degree of reduction in urine protein and serum creatinine correlated with the degree of protection from structural damage in the glomeruli, suggesting that the combination treatment results in a significantly greater protection from the permanent damage to the kidney in this disease model of human nephropathy. Thus, the results suggest that the combination treatment would have a significant effect on human nephropathic diseases involving loss of glomerular function due to multiple etiologies (e.g., diabetic nephropathy, chronic hypertension with microalbuminurea, forms of glomerulonephritis such as membranous nephropathy, and focal segmental glomerulosclerosis).

### Example 2

### Blood Pressure in Rat Remnant Kidney Model

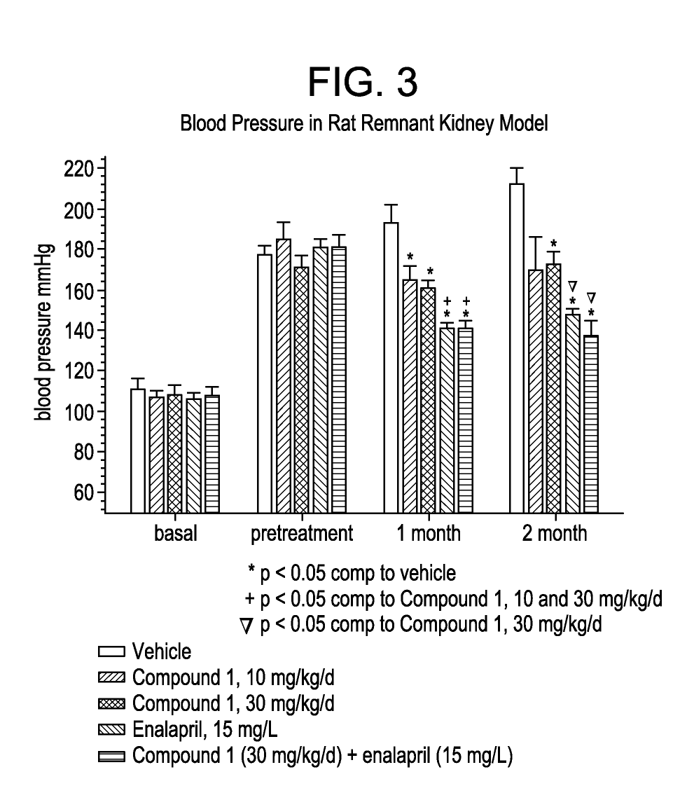

Blood pressure reduction can contribute to slowing the progression of renal disease in this animal model as well as in humans. Both enalapril and to a lesser extent Compound 1 at both 10 and 30 mg/kg/d caused significant reductions in arterial pressure compared to vehicle (FIG. 3). However, the combination of enalapril and Compound 1, 30 mg/kg/d, had blood pressure reductions comparable to enalapril alone, thus indicating no further reduction in arterial pressure with Compound 1 when given with enalapril. This indicates that the additional benefits of adding Compound 1 to enalapril were likely not the result of decreases in systemic arterial pressure but rather a direct nephroprotective effect.

### Example 3

### Effects of Water Intake and Diuresis in Remnant Kidney Model

Water intake and diuresis was increased in all treatment groups following RMR (Table 2). There were no significant differences in any of the treatment groups over the course of the experiment (Table 2). There were no differences in serum electrolytes in any of the treatment groups at the termination of the experiment (Table 2). Urine electrolytes were significantly lower in all treatment groups compared to normal control rats. However, urine chloride ion (Cl⁻) in the enalapril alone group and the Compound 1+enalapril group, and urine potassium ion (K⁺) in the Compound 1+enalapril group were significantly greater than those in the vehicle group (Table 2). There were no significant differences in food consumption or body weight among the treatment groups (data not shown).

TABLE 2
Effects on Water Intake and Diuresis in Remnant Kidney Model

| Treatment | | | Pre- | | |
|---|---|---|---|---|---|
| Group | | basal | treatment | 1 month | 2 month |
| Vehicle | water | 45 ± 4 | 76 ± 6** | 74 ± 4** | 62 ± 3** |
| | intake | | | | |
| | diuresis | 15 ± 2 | 50 ± 5** | 51 ± 3** | 48 ± 4** |
| Compound 1 | water | 43 ± 5 | 66 ± 5* | 53 ± 9° | 62 ± 6* |
| 10 mg/kg/d | intake | | | | |
| | diuresis | 19 ± 1 | 44 ± 3** | 47 ± 2** | 43 ± 5** |
| Compound 1 | water | 47 ± 3 | 76 ± 6** | 60 ± 7* | 60 ± 16* |
| 30 mg/kg/d | intake | | | | |
| | diuresis | 19 ± 1 | 55 ± 4** | 41 ± 4* | 49 ± 8** |
| Enalapril | water | 44 ± 4 | 66 ± 6** | 60 ± 4*° | 59 ± 6** |
| 15 mg/L | intake | | | | |
| | diuresis | 19 ± 1** | 46 ± 5** | 46 ± 4** | 45 ± 3** |
| Compound 1 | water | 45 ± 2 | 74 ± 7* | 62 ± 3* | 72 ± 4** |
| 30 mg/kg/d + | intake | | | | |
| Enalapril | | | | | |
| | diuresis | 20 ± 1** | 51 ± 4** | 44 ± 3** | 43 ± 3** |
| control | water | 47 ± 1 | 42 ± 3 | 42 ± 4 | 39 ± 2 |
| | intake | | | | |
| | diuresis | 20 ± 1 | 22 ± 3 | 24 ± 4 | 22 ± 1 |

Values are mean water intake and diuresis in mL/24 h ± S.E. for n = 8 animals in each group at the basal and pretreatment measurement points except enalapril alone, n = 7. See Table 1 for number of animals in each group at 1 and 2 months of treatment.
*p < 0.05,
**p < 0.01 compared to control;
° p < 0.05,
°° p < 0.01 compared to vehicle

### Example 4

### Effects on Serum and Urine Electrolytes in Remnant Kidney Model

There were no significant differences in serum electrolytes at study termination in any of the treatment groups with RMR or in comparison to the control group. Urinary Na excretion was reduced in the vehicle treated RMR rats. Both doses of Compound 1 as well as enalapril alone and the combination treatment tended to normalize urinary Na excretion to similar extents. The results suggest that Compound 1 may be inhibiting tubular Na reabsorption caused by RMR. Urinary K and Cl excretion were reduced in rats with RMR compared to control rats. Both doses of Compound 1, enalapril alone and the combination treatment tended to normalize urinary K and Cl excretion.

### Example 5

### Efficacy in Streptozotocin-Induced Diabetic Rat Model of Nephropathy

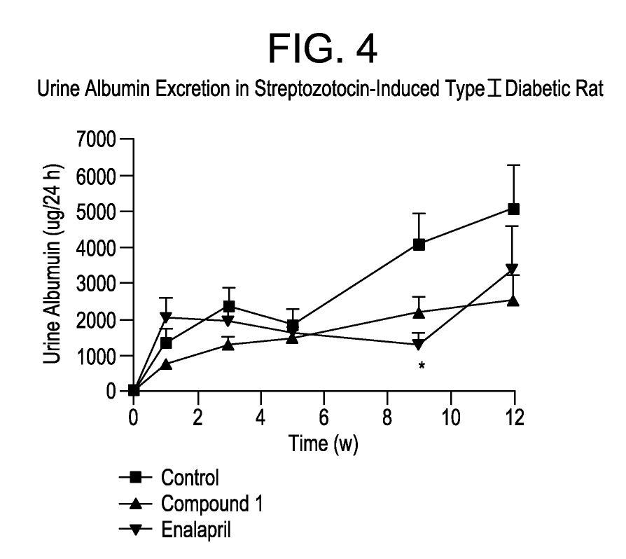

The streptozotocin-induced diabetic rat is a model of Type I diabetes that develops early renal dysfunction manifested as increased urine albumin excretion. One week after administration of streptozotocin, 65 mg/kg, i.p., rats were assigned to the following treatment groups: no treatment; Compound 1 at 30 mg/kg/d; or enalapril at 30 mg/kg/d. Both compounds were dosed in drinking water for 12 weeks. Mortality among the treatment groups at 12 weeks was 1/10 in the untreated and Compound 1-treated groups and 3/10 in the enalapril-treated group. As a result of the diabetic state, urine volumes were nearly 10 times that of non-diabetic rats. Over the course of the 12-week study, no significant treatment related effects were observed in a number of endpoints including body and spleen weight, and hematocrit. Urine endpoints including daily volume output and osmolarity as well as chloride, creatinine, sodium or potassium concentrations, clearances or excretion values were not significantly altered by either Compound 1 or enalapril treatment. A significant decrease in urine albumin excretion was observed with Compound 1 treatment compared to vehicle treated controls (FIG. 4). The fraction of filtered sodium and potassium excreted was unaffected by either Compound 1 and enalapril.

Despite an increase in kidney weight as well as glomerular and mesangial areas relative to nondiabetic controls, no significant effects on kidney weight or renal glomerular or mesangial morphology were observed with either Compound 1 or enalapril when compared to vehicle treated diabetic rats.

Although not statistically significant, serum albumin concentrations increased following Compound 1 or enalapril treatment compared to vehicle treated controls, a finding consistent with a decrease in urinary albumin excretion. Serum and urine sodium and chloride were unaltered by treatment, but a trend toward increased urine free water clearance was observed with Compound 1 treatment. Serum osmolarity decreased significantly with Compound 1 treatment (compared to vehicle treated controls). This reduction trended toward a normalization of the typically hyperosmotic serum (based on historic controls) in diabetic rats.

Oral administration of Compound 1 for seven days in Type I diabetic rats had no effect on mean arterial pressure or heart rate over 48 h in diabetic rats. In all cases, Compound 1 treated animals exhibited similar blood pressure and heart rate values across time as vehicle treated animals. Oral administration of enalapril for seven days had no effect on arterial pressure over 48 h in diabetic rat

### Example 6

### Efficacy in db/db Mouse Model of Diabetic Nephropathy

Male db/db mice, a genetic model of type 2 diabetes, develop early renal dysfunction manifested as increased urine albumin excretion. At 9 weeks of age diabetes was confirmed by measurement of blood glucose (>300 mg/dL) and animals assigned to one of three treatment groups (n=12/group): untreated, Compound 1, 30 mg/kg/d and enalapril, 30 mg/kg/d. Compounds were dosed in drinking water for 16 weeks. There was no mortality in any of the treatment groups. Body weights for animals were similar at the beginning of the study. However, mice treated with Compound 1 had increased body weight relative to the other two treatment groups from weeks 6 through 16. Body weights for the enalapril treatment group were indistinguishable from vehicle treated controls throughout the course of the study.

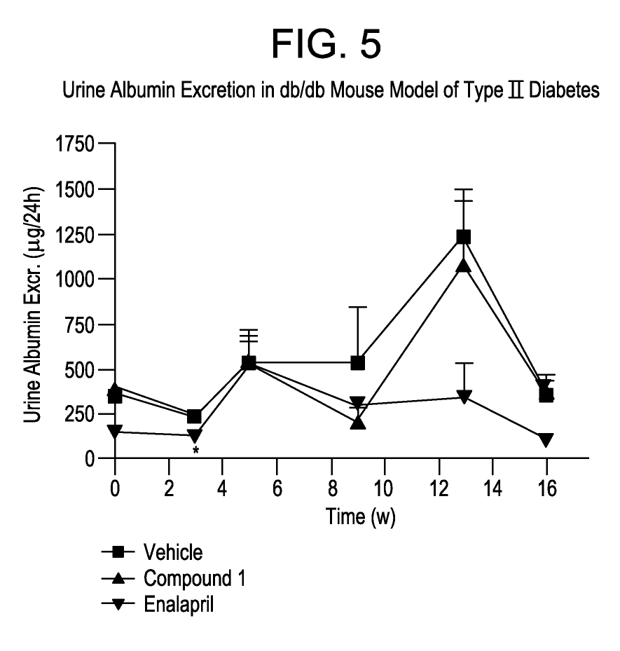

Urinary albumin concentration, the major endpoint of the study, increased in vehicle treated control animals throughout the first 12 weeks of the study, with a peak observed at the 12 week time point (FIG. 5). However, by the 16th week of vehicle dosing, vehicle-treated animals exhibited predosing albumin excretion levels (approximately 250 ug/24 h). Urinary albumin excretion was unaffected by Compound 1. However, enalapril tended to reduce urine albumin excretion with significant differences observed at 2 weeks and 16 weeks compared to vehicle controls. Enalapril tended to maintain albumin excretion in urine at or below 500 ug/24 h. Daily urine volumes tended to increase over the 16-week course of the study for all treatment groups with no differences between treatment groups observed. Urine osmolarity, measured throughout the 16 weeks of dosing with either vehicle, Compound 1 or enalapril, was unaffected by treatment.

Microscopically, glomerular surface area was not significantly different between treatment groups. Further, a separate group of age-matched control mice (dm) had glomerular surface areas comparable to those of those of all other treatment groups. Mesangial surface area and the percentage of glomerular area occupied by mesangium were significantly elevated in vehicle-treated animals compared to non-diabetic controls. No significant differences in mesangial surface area or the percentage of glomerular area occupied by mesangium were observed with Compound 1 or enalapril compared to vehicle-treated animals.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A pharmaceutical composition comprising at least one angiotensin-converting enzyme inhibitor, at least one vasopressin V1a/V2 receptor antagonist, and a pharmaceutically acceptable carrier.

2. The pharmaceutical composition of claim 1 wherein at least one angiotensin-converting enzyme inhibitor selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril and at least one vasopressin antagonist is selected from Formula (I) 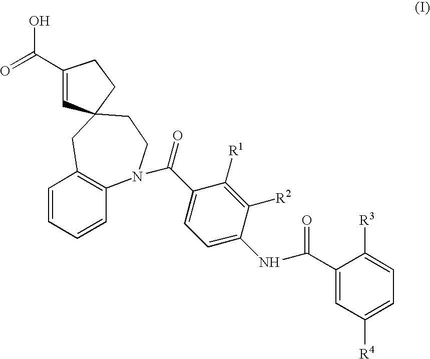
wherein
one of R¹ and R² is H and the other is H, NR⁵R⁶, C₁₋₆ alkoxy, hydroxy, or halo; wherein each of R⁵ and R⁶ is independently H or C₁₋₃ alkyl;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier.

3. The pharmaceutical composition of claim 2, wherein at least one angiotensin-converting enzyme inhibitor selected from the group consisting of enalapril and lisinopril.

4. The pharmaceutical composition of claim 3, wherein at least one angiotensin-converting enzyme inhibitor is lisinopril.

5. The pharmaceutical composition of claim 4 wherein at least one at least one vasopressin antagonist is selected from Formula (I) 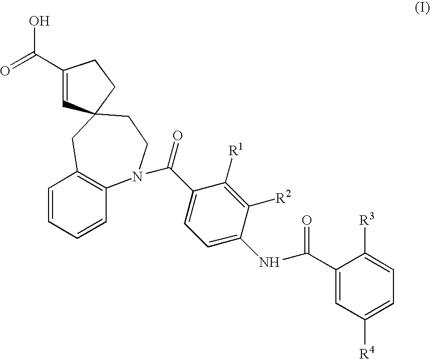
wherein
one of R¹ and R² is H and the other is C₁₋₆ alkoxy;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier.

6. The pharmaceutical composition of claim 5 wherein at least one vasopressin antagonist is 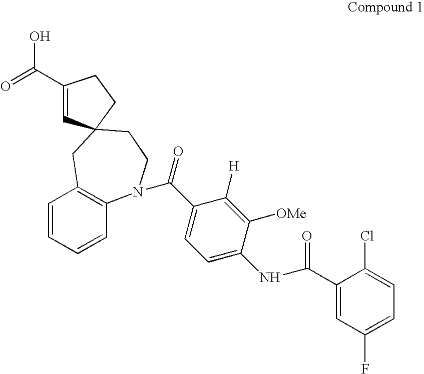
or a pharmaceutically acceptable C1-6 ester, C1-6 amide, or di(C1-6 alkyl)amide or salt thereof, and a pharmaceutically acceptable carrier.

7. A method for treating a vasopressin and/or ACE mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

8. The method according to claims 7, wherein said at least one angiotensin-converting enzyme inhibitor is selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril, in combination with at least one vasopressin antagonist selected from Formula (I) 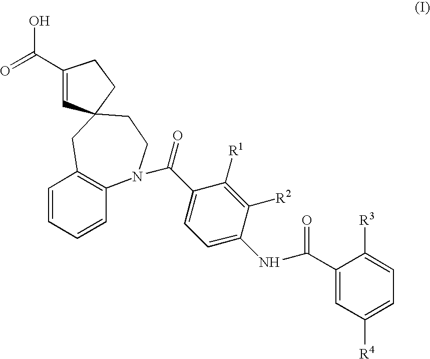
wherein
one of R¹ and R² is H and the other is H, NR⁵R⁶, C₁₋₆ alkoxy, hydroxy, or halo; wherein each of R⁵ and R⁶ is independently H or C₁₋₃ alkyl;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

9. The method according to claim 8, wherein said at least one angiotensin-converting enzyme inhibitor is selected from the group consisting of enalapril and lisinopril.

10. The method according to claim 9, wherein said at least one angiotensin-converting enzyme inhibitor is lisinopril.

11. The method according to claim 9, wherein said at least one angiotensin-converting enzyme inhibitor is enalapril.

12. The method according to claim 8, wherein said vasopressin antagonist is selected from Formula (I) 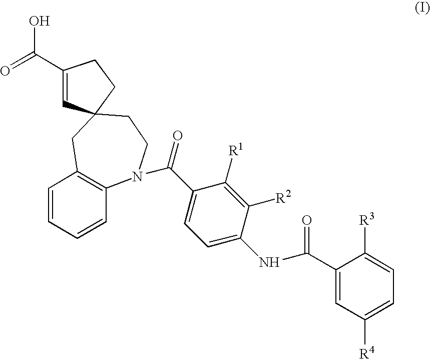
wherein
one of R¹ and R² is H and the other is C₁₋₆ alkoxy,;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

13. The method according to claim 12, wherein said at least one vasopressin antagonist is 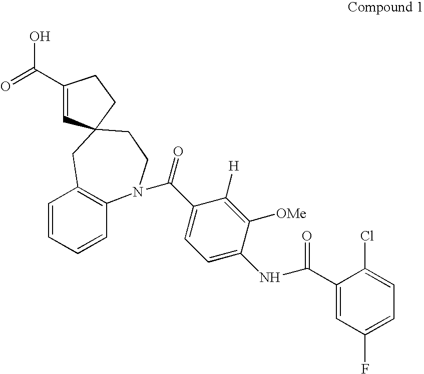
or a pharmaceutically acceptable C1-6 ester, C1-6 amide, or di(C1-6 alkyl)amide or salt thereof, and a pharmaceutically acceptable carrier.

14. A method for ameliorating a vasopressin and/or ACE mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

15. The method according to claims 14, wherein said at least one angiotensin-converting enzyme inhibitor is selected from the group consisting of captopril, enalapril, enalaprilat, lisinopril, ramipril, zofenopril, ceroanapril, alacepril, benazepril, delapril, pentopril, quinapril, quinaprilat, moexipril, rentiapril, quinapril, spirapril, cilazapril, perindopril, and fosinopril, in combination with at least one vasopressin antagonist selected from Formula (I) 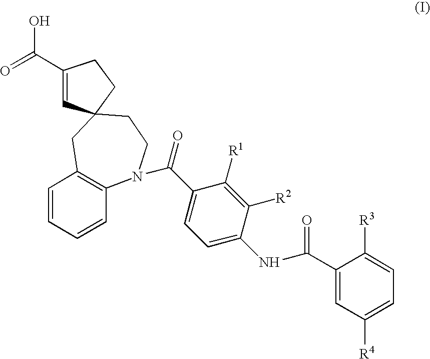
wherein
one of R¹ and R² is H and the other is H, NR⁵R⁶, C₁₋₆ alkoxy, hydroxy, or halo; wherein each of R⁵ and R⁶ is independently H or C₁₋₃ alkyl;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

16. The method according to claim 15, wherein said at least one angiotensin-converting enzyme inhibitor is selected from the group consisting of enalapril and lisinopril.

17. The method according to claim 16, wherein said at least one angiotensin-converting enzyme inhibitor is lisinopril.

18. The method according to claim 15, wherein said vasopressin antagonist is selected from Formula (I) 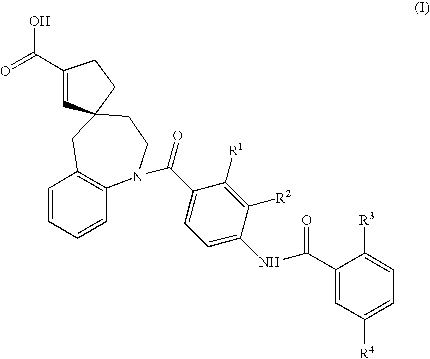
wherein
one of R¹ and R² is H and the other is C₁₋₆ alkoxy,;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

19. The method according to claim 18, wherein said at least one vasopressin antagonist is 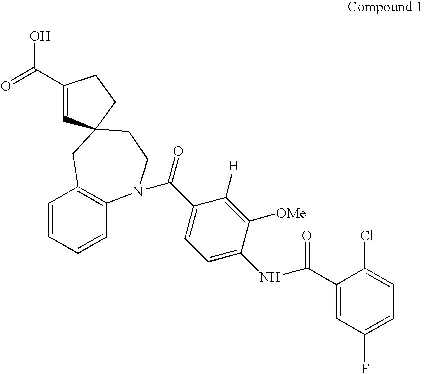
or a pharmaceutically acceptable C₁-6 ester, C1-6 amide, or di(C1-6 alkyl)amide or salt thereof, and a pharmaceutically acceptable carrier.

20. A method for preventing the progression of a vasopressin and/or ACE mediated disorder, or associated symptoms or complications thereof in a subject, said method comprising administering to said subject a therapeutically effective amount of at least one angiotensin-converting enzyme inhibitor in combination with at least one vasopressin receptor antagonist, said combined administration providing the desired therapeutic effect.

21. The method according to claims 20, wherein said at least one angiotensin-converting enzyme inhibitor is selected from the group consisting of captopril, enalapril, enalaprilat, lisinopril, ramipril, zofenopril, ceroanapril, alacepril, 5 benazepril, delapril, pentopril, quinapril, quinaprilat, moexipril, rentiapril, quinapril, spirapril, cilazapril, perindopril, and fosinopril, in combination with at least one vasopressin antagonist selected from Formula (I) 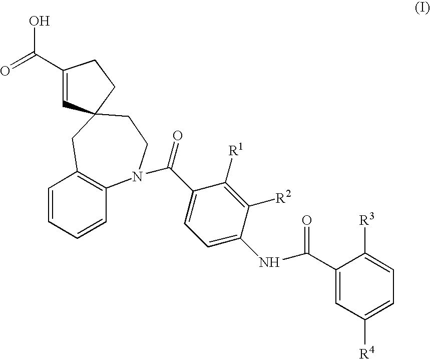
wherein
one of R¹ and R² is H and the other is H, NR⁵R⁶, C₁₋₆ alkoxy, hydroxy, or halo; wherein each of R⁵ and R⁶ is independently H or C₁₋₃ alkyl;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

22. The method according to claim 21, wherein said at least one angiotensin-converting enzyme inhibitor is selected from the group consisting of enalapril and lisinopril.

23. The method according to claim 22, wherein said at least one angiotensin-converting enzyme inhibitor is lisinopril.

24. The method according to claim 20, wherein said vasopressin antagonist is selected from Formula (I) 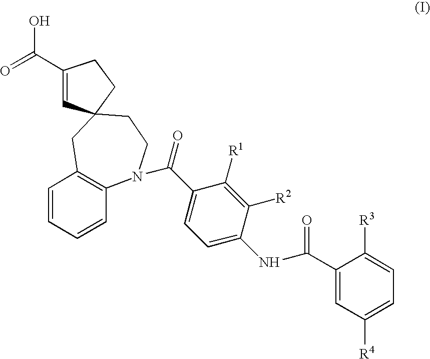
wherein
one of R¹ and R² is H and the other is C₁₋₆ alkoxy;
R³ is chloro;
R⁴ is chloro, fluoro, methoxy, or methyl;
or a pharmaceutically acceptable C₁₋₆ ester, C₁₋₆ amide, or di(C₁₋₆ alkyl)amide or salt thereof, and a pharmaceutically acceptable carrier, said combined administration providing the desired therapeutic effect.

25. The method according to claim 24, wherein said at least one vasopressin antagonist is 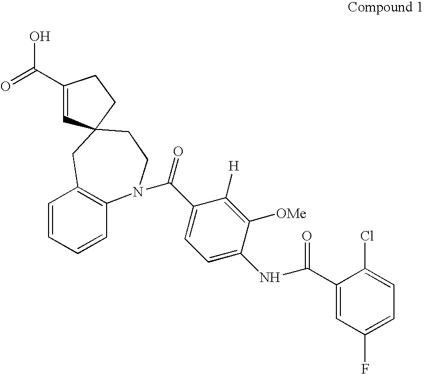
or a pharmaceutically acceptable C1-6 ester, C1-6 amide, or di(C1-6 alkyl)amide or salt thereof, and a pharmaceutically acceptable carrier.

26. The method of claim 25 wherein the disorder is selected from disease states of inner ear disorders, hypertension, congestive heart failure, cardiac insufficiency, hyponatremia, coronary vasospasm, cardiac ischemia, liver cirrhosis, renal vasospasm, renal failure, diabetic nephropathy, polycystic kidney disease, cerebral edema and ischemia, stroke, thrombosis, water retention, aggression, obsessive-compulsive disorders, dysmenorrhea, nephrotic syndrome, and central nervous injuries.

27. The method of claim 25 wherein the disorder is selected from disease states of nephropathy, and progressive renal failure (including diabetic nephropathy), polycystic kidney diseases, congestive heart failure, hypertension, diseases resulting in hyponatremia and/or edema, and other diseases resulting from excessive activation of vasopressin V1a and V2 receptors.

28. The method of claim 25 wherein the disorder is nephropathy.

29. The method of claim 25 wherein the disorder is renal failure.

30. The method of claim 25 wherein the disorder is hyponatremia.

31. The method of claim 25 wherein the disorder is polycystic kidney disease.

32. The method of claim 31 wherein said therapeutically effective amount comprises a dose range of from about 0.1 mg to about 1,000 mg.

33. The method of claim 32 wherein said therapeutically effective amount comprises a dose range of from about 50 mg to about 1000 mg.
